(19) United States
(12) Patent Application Publication
(10) Pub. No.: US 2008/0294236 A1
(43) Pub. Date: Nov. 27, 2008

(54) Endoprosthesis with Select Ceramic and Polymer Coatings

(75) Inventors: Umang Anand, Minneapolis, MN (US); Torsten Scheuermann, Munich (DE); Michael Kuehling, Munich (DE)

Correspondence Address: FISH & RICHARDSON PC, P.O. BOX 1022, MINNEAPOLIS, MN, 55440-1022, US

(73) Assignee: BOSTON SCIENTIFIC SCIMED, INC.

(21) Appl. No.: 11/752,735

(22) Filed: May 23, 2007

(51) Int. Cl. A61F 2/00

(52) U.S. Cl. 623/1.15; 427/2.24

(57) ABSTRACT

An endoprosthesis, such as a stent, includes a ceramic, such as IROX, having a select morphology and composition and a polymer coating, both of which are deposited by pulsed laser deposition.

### TECHNICAL FIELD

This disclosure relates to endoprosthesis with select ceramic and polymer coatings.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprosthesis include stents, covered stents, and stent-grafts.

Endoprosthesis can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721, the entire contents of which is hereby incorporated by reference herein.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

### SUMMARY

In a first aspect, the invention features an endoprosthesis including a ceramic layer, a polymer layer, and an interface region between the ceramic and polymer layers. The interface region composed of a composite of polymer and ceramic.

In another aspect, the invention features an endoprosthesis a composite layer of polymer and ceramic having a thickness of about 30 nm or more.

In another aspect, the invention features a method of forming an endoprosthesis, including providing a substrate, depositing a ceramic and a polymer onto the substrate by PLD, and utilizing the deposited ceramic and polymer in an endoprosthesis.

In another aspect, the invention features a method of forming an endoprosthesis including providing a substrate, depositing a ceramic onto said substrate by PLD, and utilizing the deposited ceramic in an endoprosthesis.

Embodiments may also include one or more of the following features. The polymer material in the interface region has a different molecular weight than the polymer material in the polymer layer. The polymer material in the interface region has a lower molecular weight than the polymer material in the polymer layer. The polymer material in the interface region and the polymer material in the polymer layer have the same chemical formula. The polymer material in the interface region and the polymer material in the polymer layer have different chemical formulas. The interface region has a varying relative amount of ceramic and polymer as a function of thickness. The amount of polymer increases toward the polymer layer. The interface region has a thickness of about 10 nm to 1 μm. The interface region has a thickness of about 50-100 nm. The ceramic has a globular morphology. The interface region has a thickness of about 30 nm or more.

Embodiments may also include one or more of the following features. The globular morphology has a peak height of about 20 nm or less, and a peak diameter of about 100 nm or less. The ceramic has a defined grain morphology. The defined grain morphology has a grain including a length of about 50 to 500 nm and a width of about 5 to 50 nm, and a depth of about 100 to 400 nm. The interface region has a thickness of about 300 nm or more. The ceramic has an Sdr of about 40 or more. The ceramic has an Sq of about 20 or more. The ceramic morphology of the interface region is different than the morphology of the ceramic layer. The morphology of the interface region is a defined grain morphology and the morphology of the ceramic layer is a globular morphology. The endoprosthesis is a stent including abluminal and adluminal surface regions, and wherein the ceramic layer, polymer layer, and interface region are on the abluminal surface region. The polymer layer and interface region are only on the abluminal surface region.

Embodiments may also include one or more of the following features. The adluminal region includes a ceramic layer. The ceramic layer on the abluminal surface region and the ceramic layer on the adluminal surface region have substantially the same morphology. The morphology is globular. The ceramic layer on the abluminal surface region and the ceramic layer on the adluminal surface region have different morphologies. The ceramic layer on the abluminal surface region is defined grain and the ceramic layer on the adluminal surface region is globular. The ceramic is IROX. The ceramic is on a stent body formed of metal. The metal is stainless steel. The polymer includes drug.

Embodiments may also include one or more of the following features. The ceramic has a globular morphology. The globular morphology has a peak height of about 200 nm or less, and a peak diameter of about 100 nm or less. The ceramic has a defined grain morphology. The defined grain morphology has a grain including a length of about 50 to 500 nm and a width of about 5 to 50 nm and a depth of about 100 to 400 nm. A method comprising sequentially depositing said ceramic and polymer. A method comprising depositing ceramic before depositing polymer. A method comprising simultaneously depositing said ceramic and polymer. A method comprising depositing ceramic without depositing polymer prior to simultaneously depositing. A method comprising depositing polymer without depositing ceramic after simultaneously depositing.

Embodiments may also include one or more of the following features. A polymer is applied by non-PLD after simultaneously depositing polymer and ceramic. A polymer is applied by non-PLD including applying a different polymer than the polymer in a simultaneously deposited step. A ceramic and polymer are deposited onto a substrate in a chamber without removing said substrate from the chamber. Multiple layers of ceramic and/or polymer are alternately deposited. A polymer a polymer applied without PLD is provided over a PLD-deposited polymer. The ceramic is IROX.

Embodiments may also include one or more of the following features. The ceramic has a globular morphology. The ceramic has a peak height of about 20 nm or less, and a peak diameter of about 100 nm or less and an Sdr of about 20 or less, and an Sq of about 15 or less. The ceramic has a defined grain morphology. The ceramic has a grain including a length of about 50 to 500 nm and a width of about 5 to 50 nm, and a depth of about 100 to 400 nm and an Sdr of about 40 or more, and Sq of about 20 or more.

Embodiments may include one or more of the following advantages. Stents can be formed with coatings of ceramic and polymer that have morphologies and/or compositions that enhance therapeutic performance. In particular, the ceramic and the polymer can be deposited to form an interpenetrating network that enhances the adhesion between the two materials to reduce the likelihood of flaking or delamination. The ceramics and polymers are tuned to enhance mechanical performance and physiologic effect. Enhanced mechanical performance provides particular advantages during the challenging operations encountered in stent use, which typically include collapsing the stent to a small diameter for insertion into the body, delivery though a tortuous lumen, and then expansion at a treatment site. Enhancing mechanical properties of the ceramic reduces the likelihood of cracking or flaking of the ceramic, and enhanced adhesion of the ceramic to the stent body and to overcoatings, such as drug eluting materials. Improved physiologic effects include discouraging restenosis and encouraging endothelialization. The ceramics are tuned by controlling ceramic morphology and composition. For example, the ceramic can have a morphology that enhances endothelial growth, a morphology that enhances the adhesion of overcoatings such as polymers, e.g. drug eluting coatings, a morphology that reduces delamination, cracking or peeling, and/or a morphology that enhances catalytic activity to reduce inflammation, proliferation and restenosis. The ceramics can be tuned along a continuum of their physical characteristics, chemistries, and roughness parameters to optimize function for a particular application. Different coating morphologies can be applied in different locations to enhance different functions at different locations. For example, a high roughness, low coverage, defined-grain morphology can be provided on abluminal surfaces to enhance adhesion of a drug-eluting polymer coating and a low roughness, high coverage, globular morphology can be provided on the adluminal surface to enhance endothelialization. The composition is tuned to control hydrophobicity to enhance adhesion to a stent body or a polymer and/or control catalytic effects. The morphologies and compositions can be formed by relatively low temperature deposition methodologies such as pulsed laser deposition (PLD) that allow fine tuning of the morphology characteristics and permit highly uniform, predictable coatings across a desired region of the stent. In addition, PLD can be used to deposit a polymer onto the ceramic, alternately with the ceramic, or simultaneously with the ceramic. The polymer can be used as a drug eluting polymer. A non-PLD-deposited polymer can also be bound to the PLD-deposited polymer, such that the PLD-deposited polymer is optimized for, e.g. binding to the ceramic and the non-PLD polymer is selected to optimize a therapeutic effect, e.g. drug delivery.

Still further aspects, features, embodiments, and advantages follow.

### DESCRIPTION OF DRAWINGS

FIGS. 1A-1C are longitudinal cross-sectional views illustrating delivery of a stent in a collapsed state, expansion of the stent, and deployment of the stent.

FIG. 2 is a perspective view of a stent.

FIG. 3A is a cross-sectional view of a stent wall.

FIG. 3B is a greatly enlarged cross-sectional view of a stent wall.

FIG. 4 is a schematic of a PLD system.

FIGS. 5A and 5B are enlarged plan views of a stent wall surface.

FIGS. 6A-6C are schematics of ceramic morphologies.

FIGS. 7A-7H are plan views of various morphologies.

FIG. 8 is a cross-sectional schematic of a stent wall.

FIG. 9 is a cross-sectional schematic of a stent wall.

FIGS. 10-13 are perspective views of stents.

FIG. 14 is a schematic for computing morphology parameters.

### DETAILED DESCRIPTION

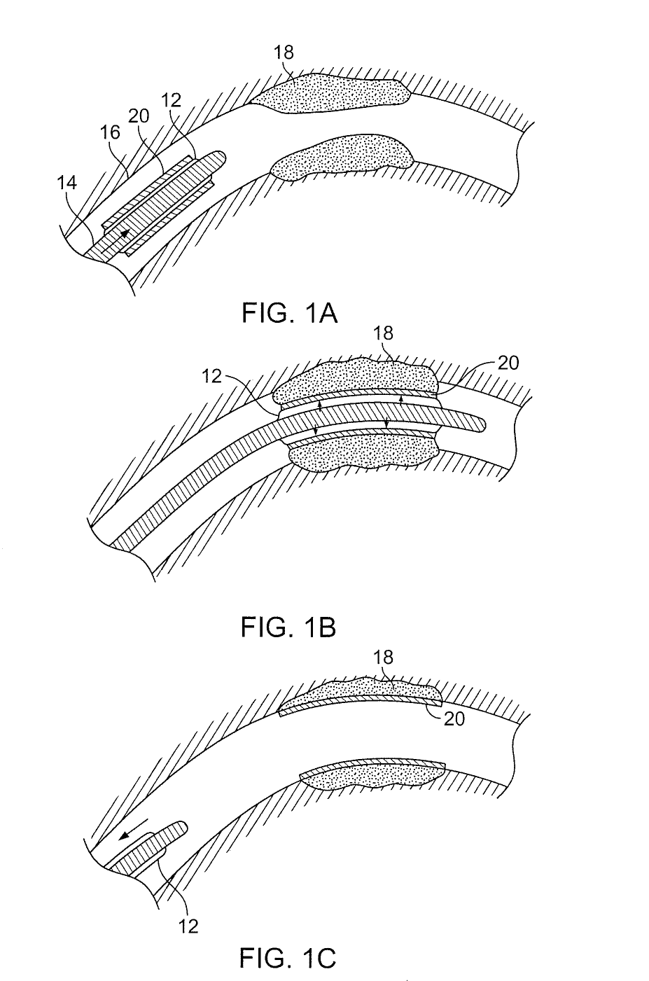

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

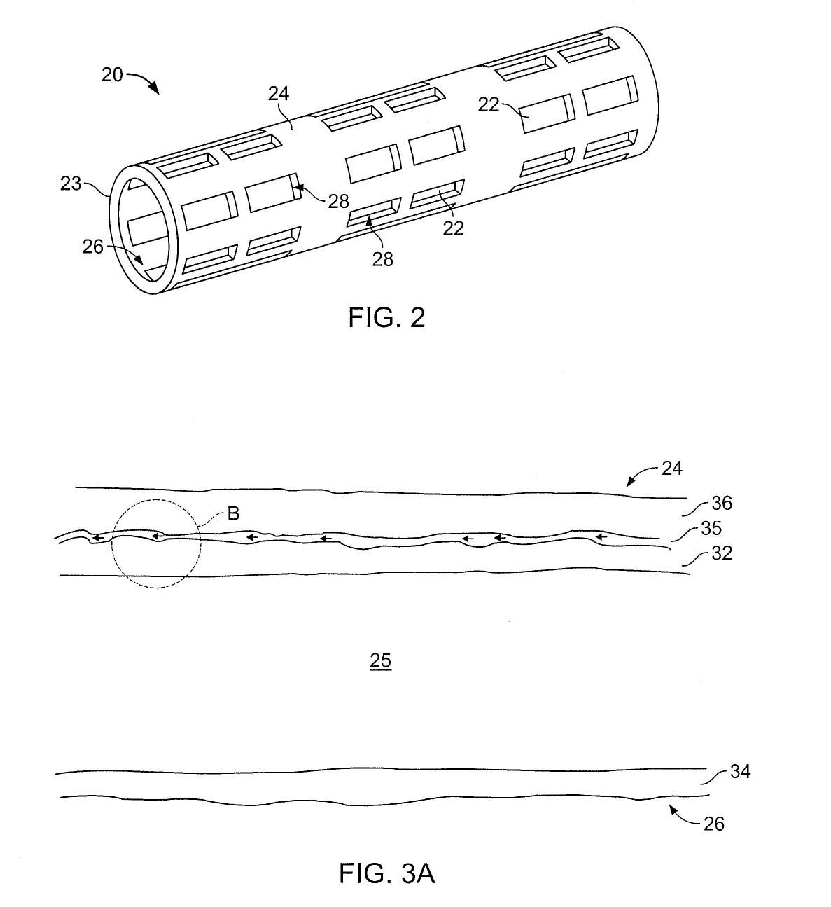

Referring to FIG. 2, the stent 20 includes a plurality of fenestrations 22 defined in a wall 23. Stent 20 includes several surface regions, including an outer, or abluminal, surface 24, an inner, adluminal, surface 26, and a plurality of cutface surfaces 28. The stent can be balloon expandable, as illustrated above, or a self-expanding stent. Examples of stents are described in Heath '721, supra.

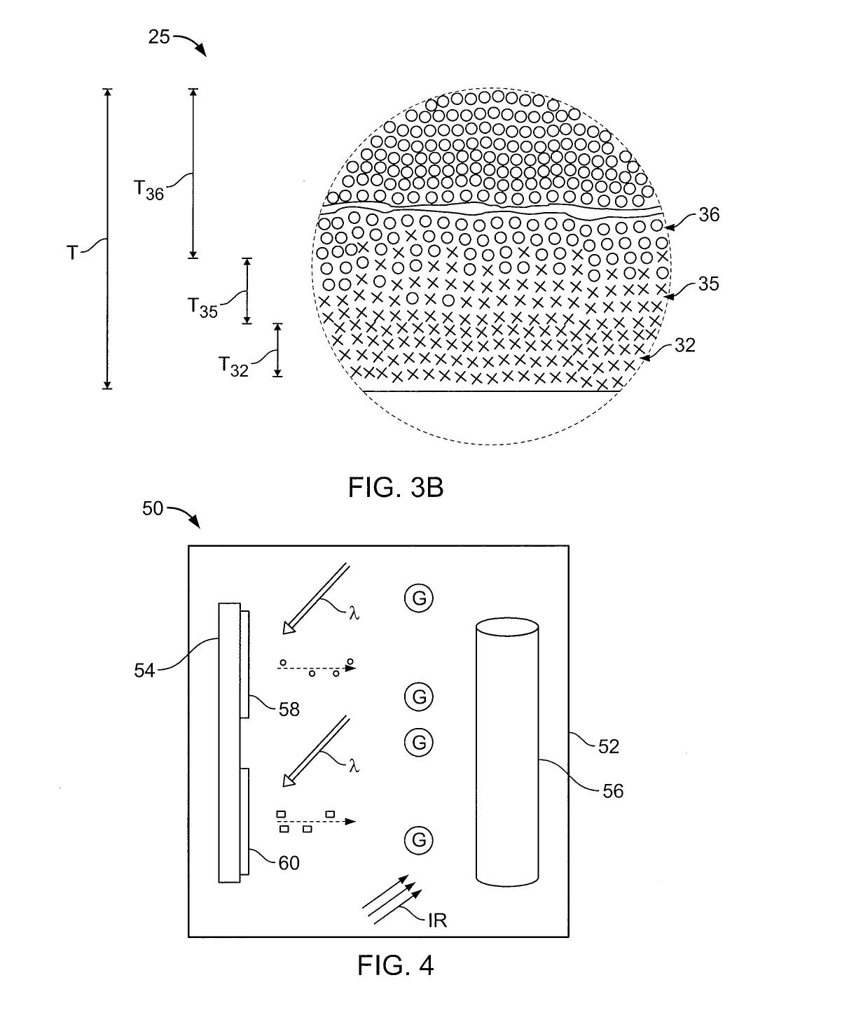

Referring to FIG. 3A, a cross-sectional view, a stent wall 23 includes a stent body 25 formed, e.g. of a metal, and includes a first ceramic coating 32 on one side, e.g. the abluminal side, and a second ceramic coating 34 on the other side, e.g. the adluminal side. The abluminal side includes a second coating 36, such as a polymer that includes a drug. The polymer coating 36 is adhered to the ceramic coating 32 along an interface region 35.

Referring to FIG. 3B, a greatly enlarged cross-section of the region B in FIG. 3A, the ceramic layer 32, with a thickness T₃₂, is deposited directly on the stent body and is composed substantially of ceramic material (X's). The polymer layer 36, having a thickness T₃₆, is composed substantially of polymer (circles). The interface region 34, having thickness T₃₄, is a mixture of ceramic and polymer. The polymer in the interface region (circles with slash) may be the same or different than the polymer in the polymer layer. In particular embodiments, the polymer in the interface region has the same chemical composition but a different, e.g. lower, molecular weight than the polymer in the polymer layer. As illustrated, in embodiments, the relative amount of ceramic and polymer in the interface region varies as a function of the distance from the stent body. Closer to the stent body, adjacent the ceramic layer, the amount of ceramic material is greater. The amount of ceramic material decreases relative to the amount of polymer closer to the polymer layer. In embodiments, the overall thickness, T, is about 1 to 10 microns, the thickness of the ceramic layer T₃₂ is about 100 nm to 500 nm, and the thickness of the polymer layer T₃₆ is about 500 nm to 2 μm. The thickness of the interface T₃₄ is about 10 nm to 1 μm, preferably greater than about 20-30 nm, e.g. 50 to 100 nm and less than about 500 nm.

Referring to FIG. 4, the ceramic and polymer are deposited by pulsed laser deposition (PLD). The PLD system 50 includes a chamber 52 in which is provided a target assembly 54 and a stent substrate 56, such as a stent body or a prestent structure such as a metal tube. The target assembly includes a first target material 58, such as a ceramic (e.g., IROX) or a precursor to a ceramic (e.g., iridium metal) and a second target material 60, such as a polymer and a gas. Laser energy (double arrows) is selectively directed onto the target materials to cause the target materials to be ablated and sputtered from the target assembly. The sputtered material is imparted with kinetic energy in the ablation process such that the material is transported within the chamber (single arrows) and deposited on the stent 56. In addition, the temperature of the deposited material can be controlled by heating, e.g. using an infrared source (triple arrows).

The composition of the deposited material is selected by control of the deposition process. For example, the composition of the deposited material is selected by controlling the exposure of the target materials to laser energy. To deposit pure polymer or pure ceramic, only the polymer or ceramic material is exposed to laser energy. To deposit a composite layer of ceramic and polymer, both materials are exposed simultaneously or alternately exposed in rapid succession. The relative amount of polymer and ceramic is controlled by the laser energy and/or exposure time. In embodiments, the ceramic and polymer are deposited as small clusters, e.g., 100 nm or less, such as 1-10 nm, and preferably smaller than the gross morphological features of the layers. In embodiments, the particles bond at contact points forming a continuous coating that is an amalgamation of the particles. In the interface region 36, polymer is bonded to ceramic to form a composite interpenetrating network, which interlocks the polymer with the ceramic to enhance adhesion of the polymer to the stent. The molecular weight of the polymer can be controlled by selecting the laser wavelength and energy. In the ablation process, energy absorbed by the target can result in cleavage of covalent bonds in polymers, such that the chain lengths and molecular weight of the polymer in the target is reduced in the deposition material. The efficiency of the cleavage process can be enhanced by selecting a laser wavelength that the polymer absorbs strongly. In addition, at higher energies, the size of ablated clusters is increased, with less overall chain cleavage.

In embodiments, a non-PLD deposited polymer can be used as an alternative to or in combination with a PLD-deposited polymer. For example, a PLD-deposited polymer can be utilized to form a composite layer of polymer interlocked to ceramic. A further layer of polymer can be applied over the composite by non-PLD techniques, such as dipping, spraying, etc. The PLD-deposited polymer acts as a binding or tie layer to the ceramic for the non-PLD polymer. The non-PLD polymer can be the same as the PLD polymer, or a different polymer. For example, the non-PLD polymer can be the same chemically as the non-PLD polymer but the non-PLD polymer can have a different, e.g., greater, molecular weight. The non-PLD polymer could also have a different chemical composition from the PLD-deposition polymer, which is selected to optimize a therapeutic effect, e.g., drug delivery. The PLD-deposited polymer is selected for its binding properties with the ceramic and the non-PLD deposited polymer.

In particular embodiments, the laser energy is produced by an excimer laser operating in the ultraviolet, e.g. at a wavelength of about 248 nm. The laser energy is about 100-700 mJ, the fluence is in the range of about 10 to 50 mJ/cm². The background pressure is in the range of about 1E-5 mbar to 1 mbar. The background gas is oxygen. The substrate temperature is also controlled. The temperature of the substrate is between 25 to 300° C. during deposition. Substrate temperature can be controlled by directing an infrared beam onto the substrate during deposition using, e.g. a halogen source. The temperature is measured by mounting a heat sensor in the beam adjacent the substrate. The temperature can be varied to control melting of the polymer and the morphology of the ceramic. The selective sputtering of the polymer or ceramic is controlled by mounting the target material on a moving assembly that can alternately bring the materials into the path of the laser. Alternatively, a beam splitter and shutter can be used to alternatively or simultaneously expose multiple materials. PLD deposition services are available from Axyntec, Augsburg, Germany. Suitable ceramics include metal oxides and nitrides, such as of iridium, zirconium, titanium, hafnium, niobium, tantalum, ruthenium, platinum and aluminum. In embodiments, the thickness of the coatings is in the range of about 50 nm to about 2 um, e.g. 100 nm to 500 nm. Iridium oxide (IROX) is discussed further in Alt, U.S. Pat. No. 5,980,566.

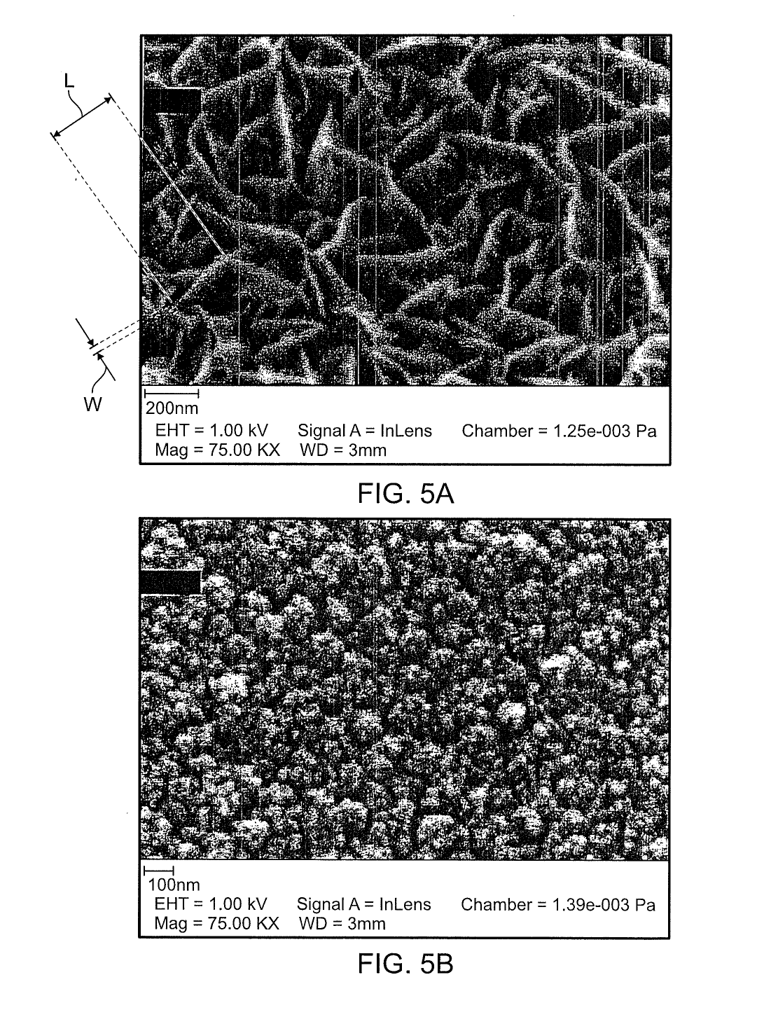

Referring to FIGS. 5A and 5B, the morphology of the ceramic can be varied between relatively rough surfaces and relatively smooth surfaces, which can each provide particular mechanical and therapeutic advantages. Referring particularly to FIG. 5A, a ceramic coating can have a morphology characterized by defined grains and high roughness. Referring particularly to FIG. 5B, a ceramic coating can have a morphology characterized by a higher coverage, globular surface of generally lower roughness. The defined grain, high roughness morphology provides a high surface area characterized by crevices between and around spaced grains into which the polymer coating can be deposited and interlock to the surface, greatly enhancing adhesion. Defined grain morphologies also allow for greater freedom of motion and are less likely to fracture as the stent is flexed in use and thus the coating resists delamination of the ceramic from an underlying surface and reduces delamination of an overlaying polymer coating. The stresses caused by flexure of the stent, during expansion or contraction of the stent or as the stent is delivered through a tortuously curved body lumen increase as a function of the distance from the stent axis. As a result, in embodiments, a morphology with defined grains is particularly desirable on abluminal regions of the stent or at other high stress points, such as the regions adjacent fenestrations which undergo greater flexure during expansion or contraction. Smoother globular surface morphology provides a surface which is tuned to facilitate endothelial growth by selection of its chemical composition and/or morphological features. Certain ceramics, e.g. oxides, can reduce restenosis through the catalytic reduction of hydrogen peroxide and other precursors to smooth muscle cell proliferation. The oxides can also encourage endothelial growth to enhance endothelialization of the stent. When a stent, is introduced into a biological environment (e.g., in vivo), one of the initial responses of the human body to the implantation of a stent, particularly into the blood vessels, is the activation of leukocytes, white blood cells which are one of the constituent elements of the circulating blood system. This activation causes a release of reactive oxygen compound production. One of the species released in this process is hydrogen peroxide, H₂O₂, which is released by neutrophil granulocytes, which constitute one of the many types of leukocytes. The presence of H₂O₂ may increase proliferation of smooth muscle cells and compromise endothelial cell function, stimulating the expression of surface binding proteins which enhance the attachment of more inflammatory cells. A ceramic, such as IROX can catalytically reduce H₂O₂. The morphology of the ceramic can enhance the catalytic effect and reduce growth of endothelial cells. Particular advantages can be realized by selecting both morphology and composition. For example, the adhesion of a polymer to a relatively smooth morphology that enhances endothelial growth can be enhanced by providing an interface region that combines polymer and ceramic in an interpenetrating network. In addition, morphology can be controlled as a function of thickness. For example, a rougher morphology ceramic can be deposited in the interface region to enhance adhesion of a polymer.

The morphology of the ceramic is controlled by controlling the energy of the sputtered particles on the stent substrate. Higher energies and higher temperatures result in defined grain, higher roughness surfaces. Higher energies are provided by increasing the temperature of the ceramic on the substrate, e.g. by heating the substrate or heating the ceramic with infrared radiation. In embodiments, defined grain morphologies are formed at temperatures of about 250° C. or greater. Globular morphologies are formed at lower temperatures, e.g. ambient temperatures without external factors. The heating enhances the formation of a more crystalline ceramic, which forms the grains. Intermediate morphologies are formed at intermediate values of these parameters. The composition of the ceramic can also be varied. For example, oxygen content can be increased by providing oxygen gas in the chamber.

The morphology of the surface of the ceramic is characterized by its visual appearance, its roughness, and/or the size and arrangement of particular morphological features such as local maxima. In embodiments, the surface is characterized by definable sub-micron sized grains. Referring particularly to FIG. 5A, for example, in embodiments, the grains have a length, L, of the of about 50 to 500 nm, e.g. about 100-300 nm, and a width, W, of about 5 nm to 50 nm, e.g. about 10-15 nm. The grains have an aspect ratio (length to width) of about 5:1 or more, e.g. 10:1 to 20:1. The grains overlap in one or more layers. The separation between grains can be about 1-50 nm. In particular embodiments, the grains resemble rice grains.

Referring particularly to FIG. 5B, in embodiments, the surface is characterized by a more continuous surface having a series of globular features separated by striations. The striations have a width of about 10 nm or less, e.g. 1 nm or less, e.g. 1 nm or about 0.1 nm. The striations can be generally randomly oriented and intersecting. The depth of the striations is about 10% or less of the thickness of the coating, e.g. about 0.1 to 5%. In embodiments, the surface resembles an orange peel. In other embodiments, the surface has characteristics between high aspect ratio definable grains and the more continuous globular surface. For example, the surface can include low aspect ratio lobes or thin planar flakes. The morphology type is visible in FESEM images at 50 KX.

The roughness of the surface is characterized by the average roughness, Sa, the root mean square roughness, Sq, and/or the developed interfacial area ratio, Sdr. The Sa and Sq parameters represent an overall measure of the texture of the surface. Sa and Sq are relatively insensitive in differentiating peaks, valleys and the spacing of the various texture features. Surfaces with different visual morphologies can have similar Sa and Sq values. For a surface type, the Sa and Sq parameters indicate significant deviations in the texture characteristics. Sdr is expressed as the percentage of additional surface area contributed by the texture as compared to an ideal plane the size of the measurement region. Sdr further differentiates surfaces of similar amplitudes and average roughness. Typically Sdr will increase with the spatial intricacy of the texture whether or not Sa changes.

In embodiments, the ceramic has a defined grain type morphology. The Sdr is about 30 or more, e.g. about 40 to 60. In addition or in the alternative, the morphology has an Sq of about 15 or more, e.g. about 20 to 30. In embodiments, the Sdr is about 100 or more and the Sq is about 15 or more. In other embodiments, the ceramic has a globular type surface morphology. The Sdr is about 20 or less, e.g. about 8 to 15. The Sq is about 15 or less, e.g. about less than 8 to 14. In still other embodiments, the ceramic has a morphology between the defined grain and the globular surface, and Sdr and Sq values between the ranges above, e.g. an Sdr of about 1 to 200 and/or an Sq of about 1 to 30.

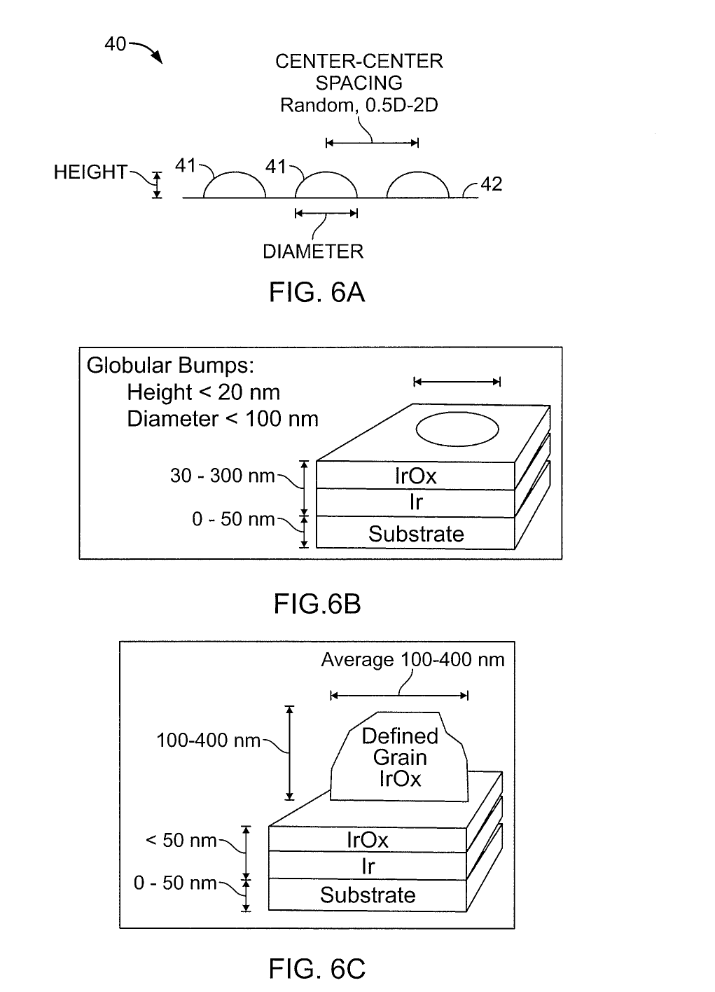

Referring to FIGS. 6A-6C, morphologies are also characterized by the size and arrangement of morphological features such as the spacing, height and width of local morphological maxima. Referring particularly to FIG. 6A, a coating 40 on a substrate 42 is characterized by the center-to-center distance and/or height, and/or diameter and/or density of local maxima. In particular embodiments, the average height, distance and diameter are in the range of about 400 nm or less, e.g. about 20-200 nm. In particular, the average center-to-center distance is about 0.5 to 2× the diameter.

Referring to FIG. 6B, in particular embodiments, the morphology type is a globular morphology, the width of local maxima is in the range of about 100 nm or less and the peak height is about 20 nm or less. In particular embodiments, the ceramic has a peak height of less than about 5 nm, e.g., about 1-5 nm, and/or a peak distance less than about 15 nm, e.g., about 10-15 nm. Referring to FIG. 6C, in embodiments, the morphology is defined as a grain type morphology. The width of local maxima is about 400 nm or less, e.g. about 100-400 nm, and the height of local maxima is about 400 nm or less, e.g. about 100-400 nm. As illustrated in FIGS. 6B and 6C, the select morphologies of the ceramic can be formed on a thin layer of substantially uniform, generally amorphous IROX, which is in turn formed on a layer of iridium metal, which is in turn deposited on a metal substrate, such as titanium or stainless steel. The spacing, height and width parameters can be calculated from AFM data. A suitable computation scheme is described below and in Appendix I.

The ceramics are also characterized by surface composition, composition as a function of depth, and crystallinity. In particular, the amounts of oxygen or nitride in the ceramic is selected for a desired catalytic effect on, e.g., the reduction of H₂O₂ in biological processes. The composition of metal oxide or nitride ceramics can be determined as a ratio of the oxide or nitride to the base metal. In particular embodiments, the ratio is about 2 to 1 or greater, e.g. about 3 to 1 or greater, indicating high oxygen content of the surface. In other embodiments, the ratio is about 1 to 1 or less, e.g. about 1 to 2 or less, indicating a relatively low oxygen composition. In particular embodiments, low oxygen content globular morphologies are formed to enhance endothelialization. In other embodiments, high oxygen content defined grain morphologies are formed, e.g., to enhance adhesion and catalytic reduction. Composition can be determined by x-ray photoelectron spectroscopy (XPS). Depth studies are conducted by XPS after FAB sputtering. The crystalline nature of the ceramic can be characterized by crystal shapes as viewed in FESEM images, or Miller indices as determined by x-ray diffraction. In embodiments, defined grain morphologies have a Miller index of <101>. Globular materials have blended amorphous and crystalline phases that vary with oxygen content. Higher oxygen content typically indicates greater crystallinity.

The morphology of the ceramic coating can exhibit high uniformity. The uniformity provides predictable, tuned therapeutic and mechanical performance of the ceramic. The uniformity of the morphology as characterized by Sa, Sq or Sdr and/or average peak spacing parameters can be within about +/−20% or less, e.g. +/−10% or less within a 1 μm square. In a given stent region, the uniformity is within about +/−10%, e.g. about +/−1%. For example, in embodiments, the ceramic exhibits high uniformity over an entire surface region of stent, such as the entire abluminal or adluminal surface, or a portion of a surface region, such as the center 25% or 50% of the surface region. The uniformity is expressed as standard deviation. Uniformity in a region of a stent can be determined by determining the average in five randomly chosen 1 μm square regions and calculating the standard deviation. Uniformity of a morphology type in a region is determined by inspection of FESEM data at 50 kx. Further discussion of ceramics and ceramic morphology and coating methods is provided in U.S. Ser. No. ______, filed concurrently [Attorney Docket No. 10527-805001].

### EXAMPLE

A series of IROX layers are formed as described in the following Table.

TABLE

| | | | | | | | | Peak | Peak |
|---|---|---|---|---|---|---|---|---|---|
| | Deposition | | | | Sq | | Uniformity | Height | Distance |
| Run | System | Parameters | | Type | nm | S_{dr} % | (FESEM) | (nm) | (nm) |
| A | PVD | — | | mixed | 33 | 25 | lower | ~44 | ~20 |
| B | PVD | — | | mixed | 12 | — | lower | | |
| C | PVD | — | | mixed | 7 | 3 | lower | ~10 | ~22 |
| D | wet | — | | mixed | 70 | 44 | lower | | |
| | chemical | | | | | | | | |
| E | cylindrical | — | | globular | 1 | 1 | high | 4-5 | 17-22 |
| | vertical | | | | | | | | |
| | magnetron | | | | | | | | |
| | sputtering | | | | | | | | |
| F | PLD | Laser wavelength: | 248 nm | globular | 10 | 12 | high | — | — |
| | | Laser energy: | 250 mJ | | | | | | |
| | | Energy density: | 2 J/cm² | | | | | | |
| | | Pulse length: | 25-45 nsec | | | | | | |
| | | Frequency: | 15-30 Hz | | | | | | |
| | | Pressure: | 0.8 mbar(O²) | | | | | | |
| | | Substrate rotation: | 1 rev/min | | | | | | |
| | | Coating time: | 1 min | | | | | | |
| | | Temperature at | ambient | | | | | | |
| | | deposition | | | | | | | |
| | | substrate: | | | | | | | |
| G | PLD | Laser wavelength: | 248 nm | defined | 23 | 55 | high | — | — |
| | | Laser energy: | 250 mJ | grain | | | | | |
| | | Energy density: | 2 J/cm² | | | | | | |
| | | Pulse length: | 25-45 nsec | | | | | | |
| | | Frequency: | 15-30 Hz | | | | | | |
| | | Pressure: | 0.8 mbar(O²) | | | | | | |
| | | Substrate rotation: | 1 rev/min | | | | | | |
| | | Coating time: | 1 min | | | | | | |
| | | Temperature at | 250° C. | | | | | | |
| | | deposition | | | | | | | |
| | | substrate: | | | | | | | |

| | | | | | | | | |
|---|---|---|---|---|---|---|---|---|
| H | closed | — | defined | 27 | 142 | high | — | — |
| | field | | grain | | | | | |
| | balanced | | | | | | | |
| | magnetron | | | | | | | |
| | sputtering | | | | | | | |

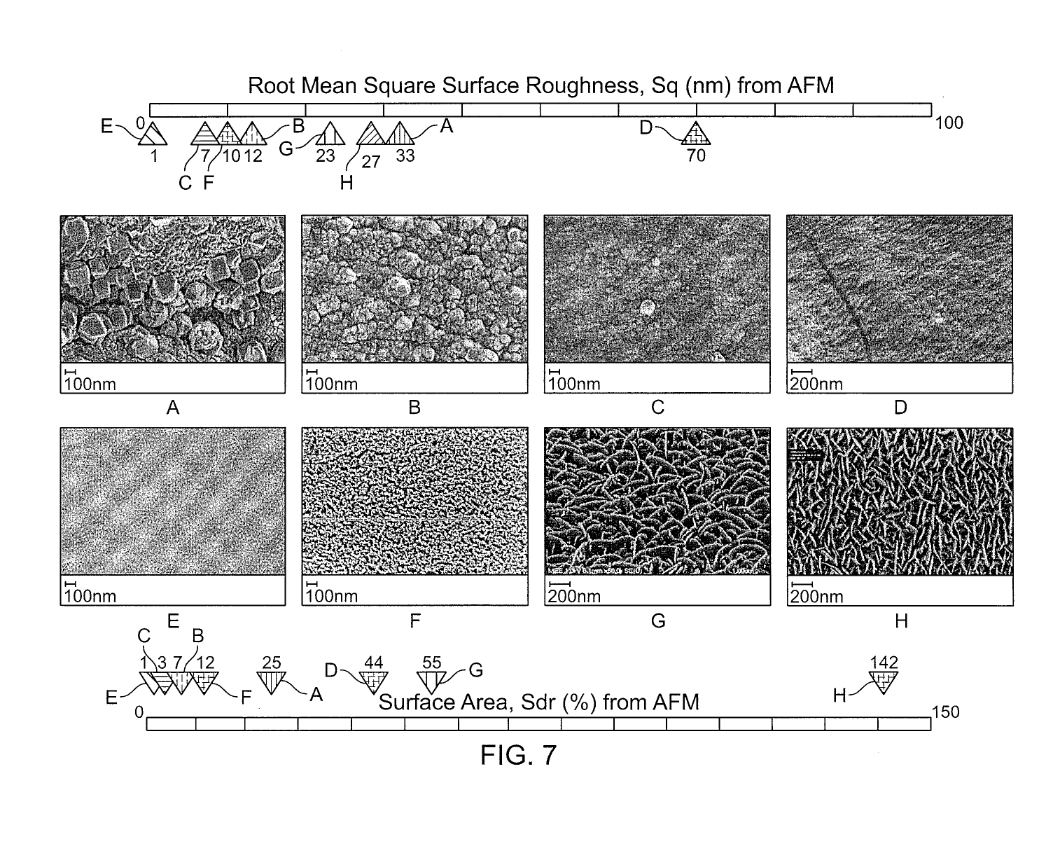

Referring to FIG. 7, FESEM images at 50K X are provided for ceramic materials formed in runs A-H, along with morphology type, Sq and Sdr values, uniformity, and peak height and distance data. Materials A-C are ceramics formed by PVD processes carried out at commercial vendors and have a surface that exhibit an intermediate morphology characterized by lobes or rock-like features integrated with or on top of smoother but still granulated surfaces. The uniformity is relatively low, with regions of lower and higher roughness and the presence of non-uniform features such as isolated regions of rock-like features. Material D is on a commercial pacemaker electrode formed by wet chemical techniques. The material exhibits a smoother but still granulated surface.

Material E is a globular material formed by cylindrical vertical magnetron sputtering. The material exhibits a relatively smooth surface, an Sq of about 1, and an Sdr of about 1. The peak height is about 4-5 nm and the peak distance is about 17-22 nm. Materials H is a defined grain material formed by closed field balanced magnetron sputtering. This material exhibits a complex, relatively rough textured surface of intersecting grains. This material has an Sq of about 27 and an Sdr of about 142. These materials exhibit high morphology uniformity.

Materials F and G are globular and defined grain materials, respectively, both of which are formed by PLD but under varying operating conditions. The defined grain material (Material G) is formed at a high substrate temperature of about 250° C. by direct infrared energy of the substrate. The globular material (Material F) is formed at lower temperature that is the ambient temperature without any external heating. Material G has an Sq of about 23 and a high Sdr of about 55. Material F has an Sq of about 10 and an Sdr of about 12.

Referring to FIG. 8, in embodiments, PLD layers are deposited sequentially and/or non PLD deposited layers are used. A stent wall 130 includes a stent body 132, a PLD deposited ceramic 134, a PLD deposited polymer 136 and a non-PLD deposited polymer 138. The PLD deposited ceramic is of a select texture, such as a globular material that enhances endothelialization. (The globular material may be provided on the opposite luminal surface as well, not shown.) The PLD deposited polymer layer 136 is a polymer suitable for drug elution or another, primer polymer compatible with both the ceramic and the polymer 138. In embodiments, the polymer layer 136 is deposited subsequently to the ceramic layer 134, but without removing the stent from the high vacuum conditions of the deposition chamber. The presence of the stent in the chamber for both processes eliminates surface contamination or stress that could result from exposure to the atmosphere. After deposition of the polymer layer 136, the stent is removed from the chamber and polymer layer 138 is applied by conventional processes, e.g., rolling or dipping. The layer 138 adheres securely to the layer 136, which is in turn strongly adhered to the ceramic layer 134.

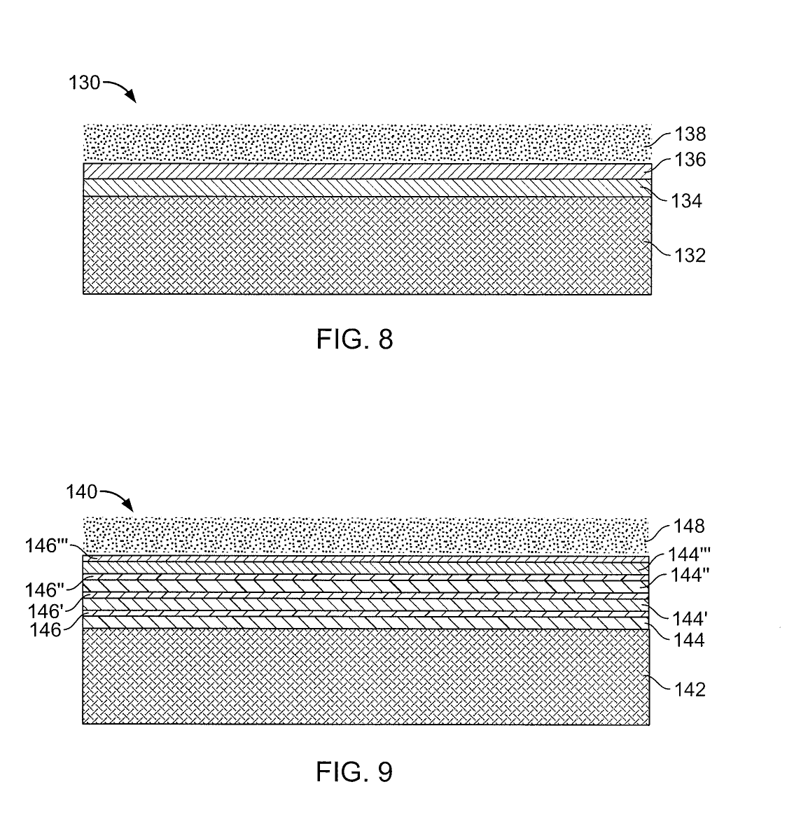

Referring to FIG. 9, a stent wall 140 includes a stent body 142, a series of PLD deposited ceramic layers 144, 144′, 144″, 144′″ and an alternating series of PLD deposited polymer layers 146, 146′, 146″, 146′″. The stent wall also includes an optional non-PLD deposited polymer layer 148. The PLD deposited ceramic layers can be of the same or different morphology, density and thicknesses. The polymer layers can be of the same or different density or thickness. For example, the layers can be very thin, e.g., about 10 nm or less. The heat generated during the PLD process, and/or heating of the substrate can melt the polymer such that it flows into interstices of the ceramic, forming an interlocked structure with high adhesion.

In embodiments, ceramic is adhered only on the abluminal surface of the stent. This construction may be accomplished by, e.g. coating the stent before forming the fenestrations. In other embodiments, ceramic is adhered only on abluminal and cutface surfaces of the stent. This construction may be accomplished by, e.g., coating a stent containing a mandrel, which shields the luminal surfaces. Masks can be used to shield portions of the stent. In embodiments, the stent metal can be stainless steel, chrome, nickel, cobalt, tantalum, superelastic alloys such as nitiniol, cobalt chromium, MP35N, and other metals. Suitable stent materials and stent designs are described in Heath '721, supra. In embodiments, the morphology and composition of the ceramic are selected to enhance adhesion to a particular metal. For example, in embodiments, the ceramic is deposited directly onto the metal surface of a stent body, e.g. a stainless steel, without the presence of an intermediate metal layer. Other suitable ceramics include metal oxides and nitrides, such as of iridium, zirconium, titanium, hafnium, niobium, tantalum, ruthenium, platinum and aluminum. The ceramic can be crystalline, partly crystalline or amorphous. The ceramic can be formed entirely of inorganic materials or a blend of inorganic and organic material (e.g. a polymer). In other embodiments, the morphologies described herein can be formed of metal. In embodiments, the thickness T of the coatings is in the range of about 50 nm to about 2 um, e.g. 100 nm to 500 nm.

As discussed above, different ceramic materials can be provided in different regions of a stent. For example, different materials may be provided on different stent surfaces. A rougher, defined grain material may be provided on the abluminal surface to, e.g. enhance adhesion of a polymer coating, while a globular material can be provided on the adluminal surface to enhance endothelialization.

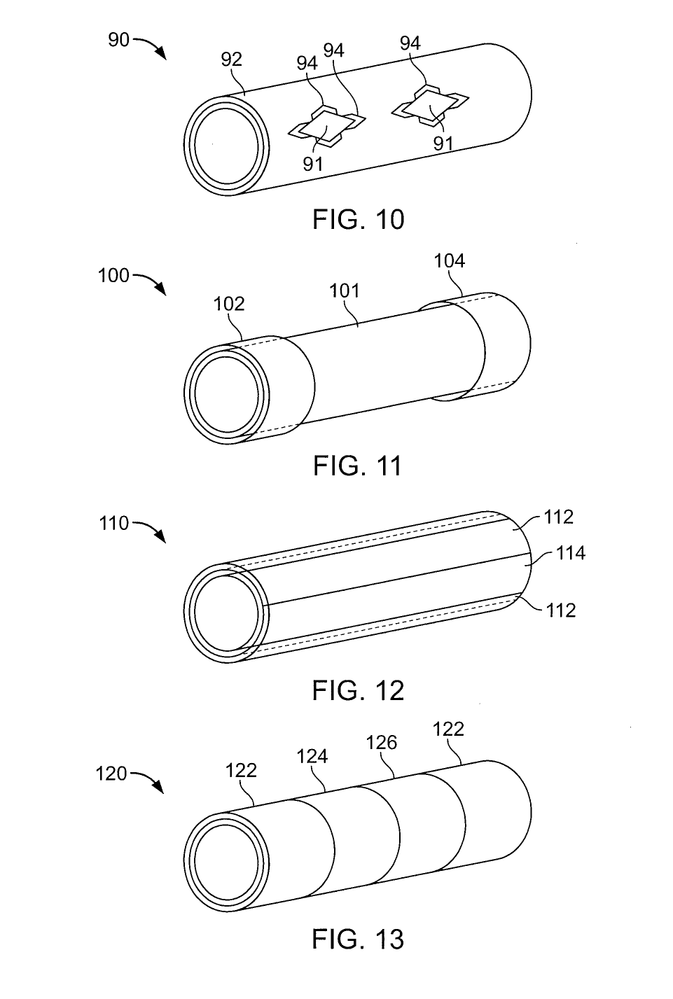

Referring to FIGS. 10-13, other patterns are illustrated. Referring to FIG. 10, a stent 90 including fenestrations 91 has first and second ceramic materials 92, 94. The ceramic material 92 covers substantially a surface of a stent except high stress regions such as adjacent to fenestrations, where material 94 is provided. Material 94 is, for example, a defined grain material that resists cracking or delamination in high stress locations and material 92 is a globular material. In embodiments, the globular material can be provided with a polymer coating, and the adhesion enhanced by forming an interpenetrating network.

Referring particularly to FIG. 11, a stent 100 includes a body 101 ceramic material 102, 104 over its end regions which correspond to the location of untreated tissue. Ceramic materials 102, 104 may be, e.g. of the same or different morphology and/or chemistry. For example, the ceramics 102, 104 can be selected to enhance endothelialization. Referring to FIG. 12, a stent 110 has a series of different ceramic materials 112, 114 arranged along its length. Referring to FIG. 13, a stent 120 has different ceramic materials 122, 124, 126 arranged radially about the stent axis. In embodiments, a polymer is provided only on the abluminal surface, as illustrated. In other embodiments, polymer layers are provided as well or only on the luminal surface and/or cut-face surfaces.

The ceramic material can also be selected for compatibility with a particular polymer coating to, e.g. enhance adhesion. For example, for a hydrophobic polymer, the surface chemistry of the ceramic is made more hydrophobic by e.g., increasing the oxygen content, which increases polar oxygen moieties, such as OH groups. Suitable drug eluting polymers may be hydrophilic or hydrophobic. Suitable polymers include, for example, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics such as polystyrene and copolymers thereof with other vinyl monomers such as isobutylene, isoprene and butadiene, for example, styrene-isobutylene-styrene (SIBS), styrene-isoprene-styrene (SIS) copolymers, styrene-butadiene-styrene (SBS) copolymers, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenerated polyalkylenes including polytetrafluoroethylene, natural and synthetic rubbers including polyisoprene, polybutadiene, polyisobutylene and copolymers thereof with other vinyl monomers such as styrene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment, the preferred polymer is polyacrylic acid, available as HYDROPLUS®. (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyiocyanates such that the devices become instantly lubricious when exposed to body fluids. In another preferred embodiment of the invention, the polymer is a copolymer of polylactic acid and polycaprolactone. Suitable polymers are discussed in U.S. Publication No. 20060038027.

The polymer is preferably capable of absorbing a substantial amount of drug solution. When applied as a coating on a medical device in accordance with the present invention, the dry polymer is typically on the order of from about 1 to about 50 microns thick, preferably about 1 to 10 microns thick, and more preferably about 2 to 5 microns. Very thin polymer coatings, e.g., of about 0.2-0.3 microns and much thicker coatings, e.g., more than 10 microns, are also possible. Multiple layers of polymer coating can be provided onto a medical device. Such multiple layers are of the same or different polymer materials.

The terms “therapeutic agent”, “pharmaceutically active agent”, “pharmaceutically active material”, “pharmaceutically active ingredient”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074. Polymers for drug elution coatings are also disclosed in U.S. Published Patent Application No. 2005/019265A. A functional molecule, e.g. an organic, drug, polymer, protein, DNA, and similar material can be incorporated into groves, pits, void spaces, and other features of the ceramic.

The stents described herein can be configured for vascular, e.g. coronary and peripheral vasculature or non-vascular lumens. For example, they can be configured for use in the esophagus or the prostate. Other lumens include biliary lumens, hepatic lumens, pancreatic lumens, uretheral lumens and ureteral lumens.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDurg 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6Al-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721). The ceramics can be used with other endoprosthesis or medical devices, such as catheters, guide wires, and filters.

Computation

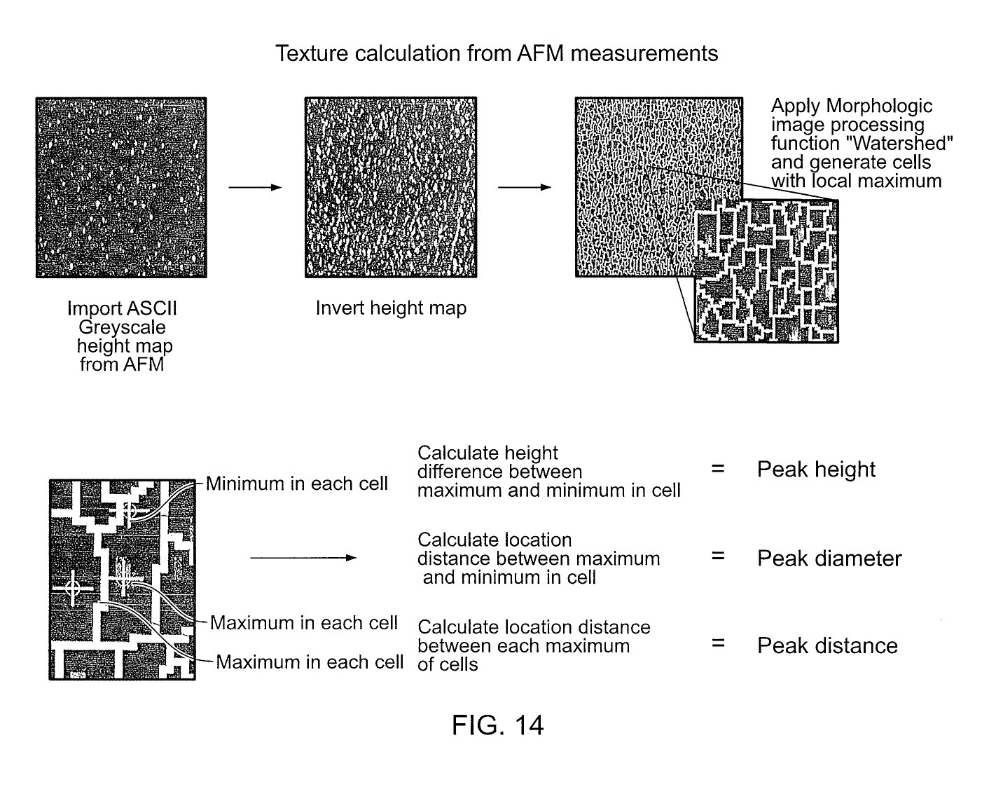

The roughness and feature parameters are calculated from AFM data. A height map is imported from the AFM as a matrix of height values. An image of a 1 um square region is represented by a 512×512 pixel matrix for a resolution of about 2-3 nm. For morphologies that exhibit substantial defined grains, the roughness parameters, Sa, Sq, and Sdr, as well as feature parameters such as peak height, peak diameter and peak distance can be calculated directly from the pixel matrix. For globular type morphologies, in which the differential between minima and maxima are less pronounced, a watershed function can be used, which is illustrated in FIG. 14. The grey scale height map is inverted and a watershed function is used to generate local maxima and minima. The roughness parameter and the feature parameter are calculated from the watershed processed data. The watershed process is used to more efficiently find local maxima and minima in the generally smoother globular morphologies. Suitable software for calculating height map is the Scanning Probe Image Processor (SPIP) from Imagnet in Lyngby, Denmark. Software for determining feature parameters is developed using IDL Software from RSI, Inc., ITT Visual Information Solutions, Boulder, Colo. Code for computing the image feature parameters is provided below.

```
  ‘
  ;  Routine for IDL Version 6.2 WIN 32 (x86)
  ;  Analyzes local peaks on height maps
  ;
  ;  xxxxxxxxxxxxxxxxxx
  ;  INPUT variables :
  ;  height_map: Matrix containing height in nm per matrix element (pixel)
  ;  pixsize: Vector containing size of one pixel in x and y direction in nm
  ;  xxxxxxxxxxxxxxxxxx
  ;
  ;  IDL is a product of ITT Visual Information Solutions www.ittvis.com
  ;  Corporate Headquarters
  ;  4990 Pearl East Circle
  ;  Boulder, CO 80301
  PRO analyze_peaks, height_map, pixsize
  ;  Calculates amount of pixels in x and y direction
  pix = {x:0,y:0}
  pix.x = (size(height_map)) [1]
  pix.y = (size(height_map)) [2]
  ;  image size x-axis in nanometers
  imagesizex = pixsize.x*pix.x/1000.0
  ;  image size y-axis in nanometers
  imagesizey = pixsize.y*pix.y/1000.0
  res = height_map
  ;shift image to positive values
  a = res − min(res)
  ;Invert the image
  b = MAX(a) − a
  c = b
  ;Create watershed image = identifies local maximum
  d = WATERSHED (c,connectivity=8)
  ;  initialize matrices
  fa = make_array(pix.x,pix.y,/float)
  fa_leveled = make_array(pix.x,pix.y,/float)
  level_max = make_array(max(d)+1,/float)
  level_min = level_max
  level_dif = level_max
  level_grad = level_max
  level_loc = level_max
  max_ix = level_max
  max_iy = level_max
  struct = make_array(2,2,/byte, value=1)
  ;  repeat calculations for each local maxima i
  for i=1, max(d) do begin
  ;  erases boundaries from identified local maximum cell
    fc = a * dilate((d eq i), struct)
    fc_min = (a−max(a)) * dilate((d eq i), struct)
    tmpa = max(fc, location_max)
    ; calculate height of local maxima for each local maxima i
    level_max[i] = tmpa
    ; calculate height of next minima for each local maxima i
    level_min[i] = min(fc_min, location_min)+max(a)
    ; calculate difference between local maxima and next minima for each maxima i
    ; = local peak height
    level_dif[i] = tmpa − level_min[i]
    fa_leveled = fa_leveled + fc − tmpa* (fc ne 0)
    ; find pixels where maxima and minima on image located
    IX_max = location_max MOD pix.x
    IY_max = location_max/pix.y
    IX_min = location_min MOD pix.x
    IY_min = location_min/pix.y
  fa[ix_max,iy_max] = 1.0
  fa[ix_min,iy_min] = −1.0
  ;  calculate lateral distance between local maxima and next minima for each local
maxima i
  ;  = local peak radius
  level_loc[i] = sqrt (((IX_max − IX_min)*pixsize.x)\hat{ }2+((IY_max −
IY_min)*pixsize.y)\hat{ }2)
  ;  calculate local maxima position
  max_ix[i] = IX_max*pixsize.x
  max_iy[i] = IY_max*pixsize.y
  ;  calculate gradient between local maxima and next minima for each local maxima i
  level_grad[i] = (level_max[i] − level_min[i])/level_loc[i]
  endfor
  ;calculate nearest distance to next local maximum
  k = 0
  k = long(k)
  maxd = long(max(d))
  max_dist1 = make_array(maxd*maxd,/float)
  ;  repeat for each permutation
  for i=1, maxd do begin
    maxdist_old =1000000.0
    for j=1, maxd do begin
      maxdist1 = sqrt ((max_ix[i] −max_ix[j])\hat{ }2+(max_iy[i]-max_iy[j]\hat{ }2)
      if (maxdist1 gt 0.0)then begin
        if maxdist1 lt maxdist_old then begin
          maxdist_old = maxdist1
        endif
      endif
    endfor
    max_dist1[i] = maxdist_old
  endfor
  ;  matrix containing distances to nearest/next local maximum = peak distances
distribution
  max_dist = max_dist1[0:maxd−1]
  ;  xxxxxxxxxxxxxxxxxxxxxxxxxxxxxxxxxxxx
  ;  results
  ;  xxxxxxxxxxxxxxxxxxxxxxxxxxxxxxxxxxxx
  ;  amount of local maxima
  print, max(d)
  ;  peak density in 1/μm\hat{ }2
  print, float(maxd)/imagesizex/ imagesizey * 1E6
  ;  average and standard deviation of nearest peak distance in nm
  print, mean(max_dist)
  print, stddev(max_dist)
  ;  average and standard deviation of local peak height in nm
  print, mean(level_diff)
  print, stddev(level_diff)
  ;  average and standard deviation of local peak diameter in nm
  print, mean(2.0*level_loc)
  print, stddev(2.0*level_loc)
  ;  average and standard deviation of local peak gradient in 1
  print, mean(level_grad)
  print, stddev(level_grad)
  End
  ;  xxxxxxxxxxxxxxxxxxxxxxxxxxx
  ;  End of routine analyze_peaks
  ;  xxxxxxxxxxxxxxxxxxxxxxxxxxx
```

All publications, patent applications, patents, and other references mentioned herein including the appendix, are incorporated by reference herein in their entirety.

Still other embodiments are in the following claims.

What is claimed is:

1. An endoprosthesis, comprising:
a ceramic layer,
a polymer layer, and
an interface region between the ceramic and polymer layers, the interface region composed of a composite of polymer and ceramic.

2. The endoprosthesis of claim 1 wherein the polymer material in the interface region has a different molecular weight than the polymer material in the polymer layer.

3. The endoprosthesis of claim 2 wherein the polymer material in the interface region has a lower molecular weight than the polymer material in the polymer layer.

4. The endoprosthesis of claim 3 wherein the polymer material in the interface region and the polymer material in the polymer layer have the same chemical formula.

5. The endoprosthesis of claim 1 wherein the polymer material in the interface region and the polymer material in the polymer layer have different chemical formulas.

6. The endoprosthesis of claim 1 wherein the interface region has a varying relative amount of ceramic and polymer as a function of thickness.

7. The endoprosthesis of claim 5 wherein the amount of polymer increases toward the polymer layer.

8. The endoprosthesis of claim 1 wherein the interface region has a thickness of about 10 nm to 1 μm.

9. The endoprosthesis of claim 1 wherein the ceramic has a globular morphology.

10. The endoprosthesis of claim 9 wherein the globular morphology has a peak height of about 20 nm or less, and a peak diameter of about 100 nm or less.

11. The endoprosthesis of claim 1 wherein the ceramic has a defined grain morphology.

12. The endoprosthesis of claim 11 wherein the defined grain morphology has a grain including a length of about 50 to 500 nm and a width of about 5 to 50 nm, and a depth of about 100 to 400 nm.

13. The endoprosthesis of claim 1 wherein the ceramic morphology of the interface region is different than the morphology of the ceramic layer.

14. The endoprosthesis of claim 13 wherein the morphology of the interface region is a defined grain morphology and the morphology of the ceramic layer is a globular morphology.

15. The endoprosthesis of claim 1 wherein the endoprosthesis is a stent including abluminal and adluminal surface regions, and wherein the ceramic layer, polymer layer, and interface region are on the abluminal surface region.

16. The endoprosthesis of claim 15 wherein the polymer layer and interface region are only on the abluminal surface region.

17. The endoprosthesis of claim 16 wherein the adluminal region includes a ceramic layer.

18. The endoprosthesis of claim 17 wherein the ceramic layer on the abluminal surface region and the ceramic layer on the adluminal surface region have substantially the same morphology.

19. The endoprosthesis of claim 18 wherein the morphology is globular.

20. The endoprosthesis of claim 17 wherein the ceramic layer on the abluminal surface region and the ceramic layer on the adluminal surface region have different morphologies.

21. The endoprosthesis of claim 20 wherein the ceramic layer on the abluminal surface region is defined grain and the ceramic layer on the adluminal surface region is globular.

22. The endoprosthesis of claim 1 wherein the ceramic is IROX.

23. The endoprosthesis of claim 1 wherein the ceramic is on a stent body formed of metal.

24. The endoprosthesis of claim 23 wherein the metal is stainless steel.

25. The endoprosthesis of claim 1 wherein the polymer includes drug.

26. An endoprosthesis, comprising:
a composite layer of polymer and ceramic, the composite layer having a thickness of about 30 to 100 nm.

27. A method of forming an endoprosthesis, comprising:
providing a substrate,
depositing a ceramic and a polymer onto said substrate by PLD, and utilizing the deposited ceramic and polymer in an endoprosthesis.

28. The method of claim 27 comprising sequentially depositing said ceramic and polymer.

29. The method of claim 28 comprising depositing ceramic before depositing polymer.

30. The method of claim 27 comprising simultaneously depositing said ceramic and polymer.

31. The method of claim 30 comprising depositing ceramic without depositing polymer prior to simultaneously depositing.

32. The method of claim 30 comprising depositing polymer without depositing ceramic after simultaneously depositing.

33. The method of claim 30 wherein applying polymer by non-PLD after simultaneously depositing polymer and ceramic.

34. The method of claim 32 comprising applying polymer by non-PLD includes applying a different polymer than the polymer in said simultaneously deposited step.

35. The method of claim 27 comprising depositing said ceramic and polymer onto said substrate in a chamber without removing said substrate from said chamber.

36. The method of claim 27 comprising alternately depositing multiple layers of ceramic and/or polymer.

37. The method of claim 27 comprising providing over said PLD-deposited polymer a polymer applied without PLD.

38. The method of claim 27 wherein said ceramic is IROX.

39. A method of forming an endoprosthesis, comprising:
providing a substrate,
depositing a ceramic onto said substrate by PLD, and
utilizing the deposited ceramic in an endoprosthesis.
